(19) United States
(12) Patent Application Publication
(10) Pub. No.: US 2024/0216141 A1
(43) Pub. Date: Jul. 4, 2024

(54) PROSTHETIC CAVAL VALVE

(71) Applicant: NVT AG, Morges (CH)

(72) Inventors: Maximilian KUETTING, Gaertringen (DE); Steffen WESTERMANN, Herrenberg (DE); Moritz BAUMGAERTNER, Herrenberg (DE)

(21) Appl. No.: 18/604,792

(22) Filed: Mar. 14, 2024

Related U.S. Application Data
(63) Continuation of application No. PCT/EP2022/075762, filed on Sep. 16, 2022.

(30) Foreign Application Priority Data
Sep. 16, 2021 (DE) 10 2021 123 950.8

(51) Int. Cl. A61F 2/24; A61F 2/07

(52) U.S. Cl. CPC A61F 2/2475; A61F 2/07; A61F 2/2403; A61F 2/2409; A61F 2/246; A61F 2230/0054; A61F 2250/0098

(57) ABSTRACT

The present invention relates to a prosthetic device for treating venous backflow, wherein the prosthetic device is configured for placement into the junction between right atrium and vena cava inferior of a patient. The prosthetic device comprises an expandable stent frame and a valve element attached to the stent frame as well as an annular positioning assembly coupled to the stent frame and extending outwardly and radially from the exterior area of the stent frame.

### CROSS-REFERENCE TO RELATED APPLICATIONS

This application is a continuation of international patent application PCT/EP2022/075762, filed on Sep. 16, 2022 designating the U.S., which international patent application has been published in English language and claims priority from German patent application 10 2021 123 950.8, filed on Sep. 16, 2021. The entire contents of these priority applications are incorporated herein by reference.

### BACKGROUND

The present invention relates to a prosthetic device for treating venous backflow, the device being configured for placement into the junction between right atrium and vena cava inferior of a patient. The present invention also relates to the use of such a prosthetic device for treating tricuspid regurgitation in a mammal.

The human heart consists of four chambers, i.e., right and left atria, and right and left ventricles. The four valves of the human heart, i.e. the aortic, mitral, tricuspid and pulmonary valves, maintain a one-way blood flow through the heart, and thus ensure that blood always flows freely in a forward direction and that there is no backward leakage.

Due to their vital function, diseased or malfunctioning heart valves are a major threat for a person's life.

Tricuspid valve regurgitation is a condition in which the valve between the two right heart chambers (right ventricle and right atrium) doesn't close properly as a consequence of which the valve does not seal and blood leaks backwards across the valve. This condition—also called valvular insufficiency—reduces the heart's pumping efficiency: When the heart contracts blood is pumped forward in the proper direction but is also forced backwards through the damaged valve. As the leak worsens, the heart has to work harder to make up for the leaky valve and less blood may flow to the rest of the body.

A malfunctioning valve can be the result of a congenital heart disease or it can occur due to valve abnormalities caused by other conditions. Although congenital causes of tricuspid insufficiency exist, most cases are due to annulus dilation and dilation of the right ventricle, and this dilation leads to a derangement of the normal anatomy and mechanics of the tricuspid valve and the muscles governing its proper function. The result is incompetence of the tricuspid valve.

The treatment of severe symptomatic tricuspid regurgitation (TR) is extremely challenging for cardiologists and cardiovascular surgeons alike. Medical therapy, consisting primarily of escalating doses of diuretics, becomes ineffective in the long term as patients develop increasing diuretic resistance because of worsening renal function. Although in the United States the use of surgery for TR has shown a slight increase during the past decade, only a small portion of eligible patients undergo surgery. This is for several reasons. As patients usually are only referred for surgery late in the disease process when they have severe end-organ compromise, the procedure can be more high risk. In addition, there can be a significant rate of late recurrence of TR post-surgery.

It is in this setting that the field of transcatheter tricuspid valve intervention has gained more interest and development during the last years. E.g., EP 3 154 474 A1 discloses a system of two prosthesis for percutaneous replacement of a tricuspid valve comprising a first heart valve prosthesis to be introduced into the superior vena cava, and a second heart valve prosthesis to be introduced into the inferior vena cava.

Similarly, WO 2016/114719 A1 discloses a prosthetic valve for treating tricuspid valve regurgitation, also comprising two prosthetic stent frame-based valves for placement in the superior vena cava and the inferior vena cava.

Despite the progress that has been made in the treatment of tricuspid regurgitation over the last years, there is still a great need for improved devices facilitating the treatment for surgeons and patients.

Thus, it is an object of the present invention to provide for a device that facilitates the treatment of tricuspid regurgitation and overcomes the drawbacks of the prior art devices and treatment methods.

### SUMMARY

According to the invention, this and other objects is solved by a prosthetic device for treating venous backflow, the prosthetic device being configured for placement into the junction between right atrium and vena cava inferior of a patient and comprising an expandable stent frame having a length l, an interior area, an exterior area, the stent frame comprising a proximal portion configured for placement in the vena cava inferior and a distal portion configured for protruding from the vena cava inferior into the right atrium, a valve element attached to the stent frame, an annular positioning assembly coupled to the stent frame and extending outwardly and radially from the exterior area of the stent frame, the annular positioning assembly comprising a plurality of outwardly radially expanding positioning members, wherein the annular positioning assembly, at the stent frame, is provided between the distal portion and the proximal portion, and is configured for positioning the distal portion of the main body in the right atrium and the proximal portion in the vena cava inferior.

The object is further solved by the use of the prosthetic device for treating tricuspid regurgitation, and a method for treating tricuspid regurgitation, the method comprising the step of placing/deploying a prosthetic device of the invention in the heart region of a patient, specifically the vena cava inferior of a patient in need of treatment of the tricuspid regurgitation, such, that the proximal portion of the prosthetic device is placed in the vena cava inferior, and that the distal portion of the prosthetic device protrudes from the vena cava inferior into the right atrium, wherein the annular positioning assembly positions the distal portion of the main body in the right atrium and the proximal portion in the vena cava inferior.

The problem underlying the invention is completely solved by this way.

With the device according to the invention and its use in the treatment of tricuspid insufficiency, it is possible to securely and conveniently replace the tricuspid valve's function and, as a consequence, effectively support the heart's function.

The device according to the invention focusses on the abolition of backflow into the vena cava inferior to address the symptoms of tricuspid regurgitation mainly caused by high pulsating venous pressures affecting renal and hepatic function.

According to a preferred embodiment, the annular positioning assembly further comprises a cover member covering the positioning members.

In addition, the plurality of outwardly radially expanding positioning members, eventually together with the cover member covering the positioning members, effect a sealing of the valve, thus preventing paravalvular leaking.

In this context, an “annular” positioning assembly means, that the assembly is circumferentially provided at the stent frame, such, that the plurality of outwardly radially expanding positioning members circumferentially extend from the outer surface of the stent frame at a certain angle and at a certain distance from one another.

According to a preferred embodiment of the invention, the valve element is attached to the stent frame in the distal portion.

With the device of the invention, the time and surgical expense of the valve replacement procedure can be efficiently improved, because the annular positioning assembly allows for intuitive placement and safe anchoring of the device. The annular positioning assembly, which is coupled to the stent frame, by extending outwardly and radially from the exterior of the stent frame, guarantees and effects the secure positioning and anchoring of the device at the desired position, i.e. such, that the proximal portion of the prosthetic device is placed in the vena cava inferior, and that the distal portion of the prosthetic device protrudes from the vena cava inferior into the right atrium.

Presently, and as generally understood, a “expandable stent frame” is to be understood and referred to as a radially-expandable metal frame or body, having a lumen formed there through, and which can have a cylindrical or tubular form, however, which form does not need to be uniformly cylindrical or tubular, but which can have different diameters and shapes along its length, but which forms a lumen for guiding blood there through.

Generally, a “stent frame” means any device or structure that adds rigidity, expansion force, or support to the prosthesis. In cases where the stent is covered with a prosthesis material forming a fluid-tight or substantially fluid-tight covering for the stent frame, these devices are designated as “stent grafts”. The body of stents/stent grafts is inserted into the vessel/organ to be treated, i.e. the heart, and is expanded or self-expandable and fixed or fixes itself at the appropriate site in order to keep open the lumen of the heart. Accordingly, a “graft” material is a cylindrical liner that may be disposed on the stent's interior, exterior or both. A wide variety of attachment mechanisms are available to join the stent and graft together, including but not limited to, sutures, adhesive bonding, heat welding, and ultrasonic welding. Presently, a “covering” also may designate or is designating a graft material attached to a stent member, which is why a “stent graft” is presently, and throughout the relevant field, also designated as “covered stent” or “covered stent graft”.

The stent frame of the device according to the invention may be laser cut or woven or braided or knitted metal mesh or can comprise an otherwise interconnected metal mesh.

A stent frame can also comprise, for example, a series of singular stent elements/rings interconnected with one another via legs or struts, or, respectively, a wire framework made of a self-expanding material. The stent frame or the wire framework forms an at least partially tubular body that supports the vessel walls.

The stent frame may also represent a metal frame formed single metal rings forming a metal mesh, the rings meandering circumferentially and being disposed successively in the graft member's longitudinal axis/direction, wherein the metal rings have a Z-shaped profile with pointed arches pointing alternately toward the proximal end and distal end of the device, the single metal rings being indirectly connected with one another via a graft material covering the stent elements/rings.

According to one embodiment of the prosthetic device of the invention, the positioning assembly is attached to the stent frame, such, that the portion lying upstream of the positioning assembly is the distal portion, and that the portion lying downstream of the positioning assembly is the proximal portion. As such, the positioning assembly is “dividing” the stent frame in a distal and a proximal portion, or, in other words, represents the border between the proximal and the distal portion.

In one embodiment, the distal portion takes up from about 50% to about 5% of the total length of the stent frame, wherein the position of the positioning assembly at the stent frame representing the border between the proximal and the distal portion.

According to one embodiment of the prosthetic device of the invention, the distal portion is shorter than the proximal portion and the positioning assembly radially extending from the exterior of the stent frame is formed by portions of very distal end of the stent frame bent outwardly. In this embodiment, the distal portion takes up about between 50% to about 5%, preferably about between 30% or 20% to about 5%, of the total length of the stent frame, so that only this distal portion is positioned in the right atrium and secured there via the bent portions of the distal stent frame portion.

In yet another embodiment, the positioning assembly is positioned at the stent frame, such, that the proximal portion takes up about 70 to about 30%, preferably about 60% to about 40%, of the total length of the stent frame, with the distal portion representing about 30% to about 70%, respectively, wherein preferably both, the proximal and the distal portion each represent 50% of the total length of the stent frame, and that the positioning assembly is positioned at about the middle portion of the total length of the stent frame, or, in other words, via its position at the stent frame, “dividing” the stent frame in the proximal and the distal portion to achieve the above percentages.

Accordingly, in one embodiment, the stent frame of the prosthetic device of the invention has an at least partially substantially tubular stent frame, wherein preferably at least the proximal portion of the stent frame is tubular.

Herein, the expression “substantially” refers to a great extent or degree, e.g. “substantially tubular”, which in the context of this invention means that the form is generally tubular, but not necessarily exactly tubular, i.e. cylindrical. It means that the tubular form can have diameter variances along its length, but still retains a generally tubular form.

According to one embodiment, the proximal portion and the distal portion of the stent frame are generally tubular.

According to one aspect of the invention, in the prosthetic device of the invention the valve element comprises one or two leaflets, a valve skirt, and a plurality of valve commissure points.

Thus, in one embodiment, the valve can be formed to comprise only one leaflet, thus representing a monoleaflet-valve. A “monoleaflet” valve is generally composed of a single disk-like sheet secured to/by the metal struts of the stent frame.

On the other hand, and in another embodiment, the valve can be made of two (semilunar) disk-like sheets attached to the stent frame, thus representing a bileaflet valve.

The valve of the prosthetic device according to the invention is preferably made or composed of human or animal pericardium, or from native valves, or veins or similar.

A “valve skirt”, herein, as well as generally understood, means a valve material or prosthetic material being attached to the inside and/or the outside of the stent frame, and covering the stent frame over a certain length of the stent frame, usually only to a certain height/length of the stent frame.

“Valve commissure points” are areas where the valve leaflets abut. They also represent the points where the distal ends of the leaflets are attached to the support structure, i.e. stent frame, of the valve.

According to one aspect of the invention, in the prosthetic device of the invention, the valve element has an asymmetrical leaflet design.

In this context, “asymmetrical” means that the sheet/disk valve element is made of or has parts that fail to correspond to one another in shape, size, or arrangement, and, thus, lacks symmetry.

An asymmetrical leaflet design of the valve element can further enhance the performance at the valve position. This is achieved by enhancing the coaptation function and reducing the closing volume. This is the case specifically in the flow field of the right atrium in patients with tricuspid regurgitation, where the regurgitation jet meets the valve leaflets laterally.

In one embodiment, in the prosthetic device of the invention, the valve element comprises one valve leaflet and a flexible coaptation section, a valve skirt, and two valve commissure points.

In this context, a “flexible coaptation section” means the zone of the leaflet(s) where the surface of leaflet(s) coapt(s) or close(s), wherein the closure is flexible in that a smooth coaptation can be achieved.

This embodiment has the advantage that only one leaflet performs the opening and closing motion so that the overall material present in the prosthetic device can be reduced. This furthermore enhances the coaptation and reduces the closing volume of the valve, thereby improving the flow field and the hemodynamics.

In another embodiment of the prosthetic device of the invention, the valve element consists of one rigid leaflet and one flexible leaflet.

This embodiment has the advantage that the flexible leaflet can coapt to/with the rigid leaflet, with the flexibility of the one leaflet, and the rigidity of the other leaflet providing for a sufficient and fit closure.

According to yet another embodiment of the prosthetic device of the invention the valve element is attached within the interior area of the stent frame in the distal portion.

In another preferred embodiment, the valve commissure points are attached to one or more stent wire extensions, preferably 1, 2, or 3 stent wire extensions, extending beyond the distal end of the stent frame.

In one embodiment of the prosthetic device of the invention, the outwardly radially expanding positioning members of the positioning assembly are stent wire members selected from struts or V-shaped wire portions.

With the struts or V-shaped wire portions radially extending outwardly from the stent frame, an efficient positioning of the device at the desired position in the heart is achieved. By “V-shaped” a form is meant that resembles the form of the letter V, having an end where the two legs are connected and an end where the two legs are apart from one another. In one embodiment of the prosthetic device of the invention, the V-shape of the positioning members are arranged at the stent frame, such, that the ends of the V-shape with the connected legs points outwardly, and the other end is attached or part of the stent frame, optionally forming a closed end via the connection with/at the stent frame.

With “V-shaped” every shape is meant that resembles the letter V, however, the legs of the V can be curved outwardly to some extend or can be straight.

In an embodiment of the prosthetic device of the invention, annular positioning assembly comprises between 1 and 30, preferably between 3 and 15, outwardly radially expanding positioning members, i.e. preferably, 3, 4, 5, 6, 7, 8, 9, 10, 11, 12, 13, 14, or 15.

The number of the positioning members, which are preferably struts or V-shaped or other singularly formed, radially extending wire structures, will depend on the size of the stent frame, the dimensions of the heart, in particular of the vena cava inferior and the right atrium, and can be assessed by the treating surgeon considering these circumstances.

In one embodiment of the prosthetic device of the invention, the positioning members extend outwardly from the stent frame at an angle between 60 and 150 degrees, preferably between 90 and 120 degrees, with respect to the length of the stent frame.

With the diameter and angle of the outwardly extending positioning members, e.g. struts or V-shaped wire structures, the respective anatomical dimensions and requirements of a heart to be treated can be reflected. The specific suitable diameter and angle can be determined by a surgeon considering these circumstances.

The length of the struts can be between about 5 mm to about 20 mm, i.e. preferably about 5 to 15 mm, and preferably about 5, 6, 7, 8, 9, 10, 11, 12, 13, 14, 15, 16, 17, 18, 19, or 20 mm.

With the cover member covering the positioning members, a circumferential rim-element is formed/present at the stent frame, which protrudes at a certain angle from the longitudinal axis of the stent frame.

In one embodiment of the prosthetic device of the invention, the positioning members radially extend non-symmetrically.

While also a symmetrically, i.e. uniform extension of the positioning members (with the same length and angle in respect to the stent frame) can be provided, in certain embodiments it can be advantageous to have the positioning members asymmetrically extended from the stent frame, such, that they extend at a different angle and/or with different length from the stent frame. Also in this regard, the specific circumstances of the arrangement of the positioning members will depend on the specific anatomical requirements. With a non-symmetrical arrangement of the positioning members, an even more specific adaption to the anatomy of the heart can be achieved.

In an embodiment of the prosthetic device of the invention, the stent frame is at least partially covered on its exterior area by a prosthetic material.

With this embodiment, the guidance of the blood flow into the device is supported and a paravalvular leakage can be further prevented.

According to one embodiment of the prosthetic device of the invention, the stent frame is covered on its exterior area by a prosthetic material in the proximal portion of the stent frame only.

This embodiment has the advantage that the distal portion of the stent frame, which is positioned in the right atrium, is free of prosthetic material, thus ensuring a free blood flow in the atrium.

In an embodiment of the prosthetic device of the invention, the stent frame is at least partially covered on its exterior area by a prosthetic material, such, that the height of the covered section varies around the circumference of the stent frame.

This embodiment has the advantage that also with the covering the special, individual circumstances of the anatomy of a patient's heart can be considered.

In an embodiment of the prosthetic device of the invention, the positioning members have two faces, one face facing towards the distal portion, one face facing towards the proximal portion, and in that the cover member covering the positioning members is attached to the positioning members on either or both of the two faces of the positioning members.

Thus, the positioning members, which can be, e.g., V-shaped wire structures extending from the stent frame, have two “sides” or “planes”, with either being covered in this embodiment or both being covered. With this embodiment, an even better prevention of a leakage past the device can be achieved.

In another embodiment of the prosthetic device the invention, the cover member covering the positioning members comprises or consists of a material selected from a biological source material or a synthetic material, wherein preferably material is selected from human or animal pericardial tissue, polyester, polyurethane, polystyrene, polytetrafluorethylene, ultra-high-molecular-weight polyethylene(UHMPE), and mixtures thereof.

In one embodiment of the prosthetic device of the invention, the device comprises radiopaque markers, preferably placed in an asymmetric order onto the stent frame or onto the cover member.

Via the radiopaque markers the device can be placed under optical control at the desired position of the heart to be treated.

The prosthetic device of the invention can be either surgically implanted or delivered by transcatheter methods. When delivered via transcatheter method, the prosthetic device of the invention, in a first step, is loaded onto a suitable deployment catheter, and compressed by a retractable sheath or tube of the deployment catheter. This arrangement, i.e. the compressed prosthetic device loaded on the deployment catheter, is inserted into the heart of a patient whose tricuspid valve needs replacement or support and advanced, e.g., via the jugular vein into the vena cava superior into the right atrium and into the vena cava inferior, as far as the distal portion of the stent frame is placed into the vena cava inferior, with the distal portion protruding from the vena cava inferior into the right atrium, and with the positioning members positioning/anchoring the distal portion of the main body in the right atrium and the proximal portion in the vena cava inferior. Alternatively, the deployment catheter having the prosthetic device according to the invention loaded thereon in a compressed state can be advanced via the femoral vein into the vena cava inferior and into the right atrium, as far as the distal portion is placed into the right atrium, and the proximal portion being placed in the vena cava inferior. Correct placement can be monitored via the radiopaque markers or other visualization elements.

Upon correct placement, the sheath or the otherwise compressing means is retracted to stepwise release the prosthetic device of the invention, upon which action the positioning members of the positioning assembly device can expand and fixate the prosthetic device at the desired place between the vena cava inferior and the right atrium.

With the release of the device, the valve element mounted on the stent frame can operate as soon as the compressing means are retracted.

Further advantages and features of the invention are set forth in the following description and in the attached figures.

It will be understood that the aforementioned features and the features still to be explained below can be used not only in the respectively specified combination but also in other combinations or on their own, without departing from the scope of the present invention.

### BRIEF DESCRIPTION OF THE DRAWINGS

The aforementioned features of the invention and the features still to be explained below are shown in the figures, in which:

FIG. 1 shows a schematic drawing of a human heart;

FIG. 2 shows a schematic drawing of an exemplary embodiment of the device of the invention placed in the correct position in the heart of FIG. 1; A) in a perspective view; B) in a side view, and C) in top view; and

FIG. 3 shows a schematic drawing of a first embodiment of the prosthetic device of the invention, in the expanded state/form and outside the heart and in enlarged detail; A) in a first side perspective; B) from above; C) an asymmetrical leaflet design; and

FIG. 4 shows a schematic drawing of a second embodiment of the prosthetic device of the invention, with a short distal portion and a V-shaped design of the positioning members of the positioning assembly in the expanded state/form and outside the heart and in enlarged detail; A) in a first side perspective; B) from above.

### EMBODIMENTS

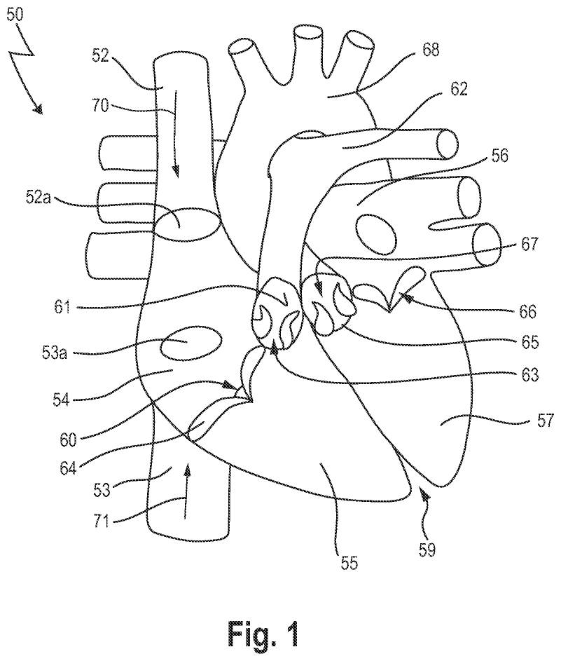

FIG. 1 shows a human heart 50, comprising a right atrium 54, a right ventricle 55, a left atrium 56 and a left ventricle 57. Also shown in FIG. 1 is a portion of the vena cava superior 52, entering the heart 50 via the right atrium 54, and a portion of the vena cava inferior 53.

The superior vena cava 52 returns the blood from the upper half of the body and opens into the upper and back part of the right atrium 54, the direction of its orifice 52a being downward and forward. Its orifice 52a has no valve.

The inferior vena cava 53, which commonly has a larger diameter than the superior vena cava 52, returns the blood from the lower half of the body, and opens into the lowest part of the right atrium 54, its orifice 53a being directed upward and backward, and guarded by a rudimentary valve, the valve of the inferior vena cava (Eustachian valve, not shown).

The right ventricle 55 has a triangular form, and extends from the right atrium 54 to near the apex 59 of the heart 50.

The right atrioventricular orifice (not depicted in FIG. 1) is the large oval aperture of communication between the right atrium 54 and ventricle 55, and is guarded by the tricuspid valve 60.

The opening 61 of the pulmonary artery 62 is circular in form, and is placed above and to the left of the atrioventricular opening; it is guarded by the pulmonary valve 63.

The tricuspid valve 60 consists of three about triangular cusps or segments or leaflets 64, the anterior, posterior and medial or septal cusp. Their bases are attached to a fibrous ring (not depicted in FIG. 1) surrounding the atrioventricular orifice and are also joined to each other so as to form a continuous annular membrane. Their atrial surfaces are directed toward the blood current from the atrium 54, while their ventricular surfaces are directed toward the wall of the ventricle 55; together with the apices and margins of the cusps, they give attachment for the chordae tendineae (not depicted in FIG. 1).

As discussed above, the function of the tricuspid valve is to prevent back flow of blood into the right atrium 54; arrows 70 and 71 indicate normal blood flow into the right atrium 54.

The left atrium 56 is smaller than the right atrium 54. The left ventricle 57 is longer and more conical in shape than the right ventricle 55. The left atrioventricular opening (mitral orifice, not depicted in FIG. 1) is placed to the left of the aortic orifice 65, and is guarded by the bicuspid or mitral valve 66.

The aortic opening 65 is a circular aperture, in front and to the right of the atrioventricular opening, and its orifice is guarded by the three aortic valves 67. Reference number 68 designates the aorta.

Tricuspid regurgitation is not uncommon in the tricuspid valve 60, and means that blood from the right ventricle 55 flows back into the right atrium 54 upon contraction of the right ventricle 55 due to the tricuspid valve 60 not properly closing.

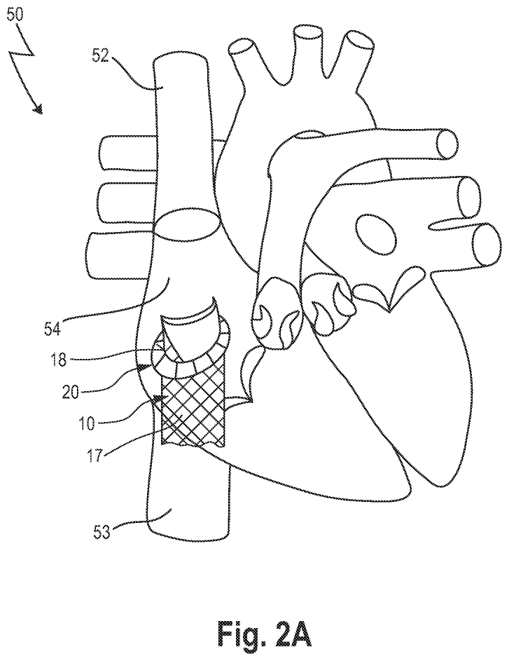

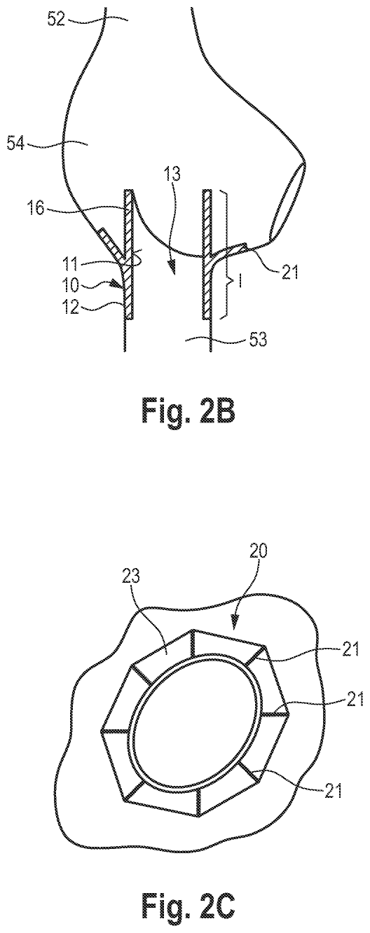

With the device according to the invention, tricuspid regurgitation is to be treated, and placement of an exemplary embodiment of the device according to the invention is schematically depicted in FIG. 2.

FIG. 2 shows the schematic drawing of the heart as already depicted in FIG. 1. For better understanding, FIG. 2 does not include all of the reference numbers as designated in FIG. 1, but is meant to show the same features of the human heart 50 as FIG. 1.

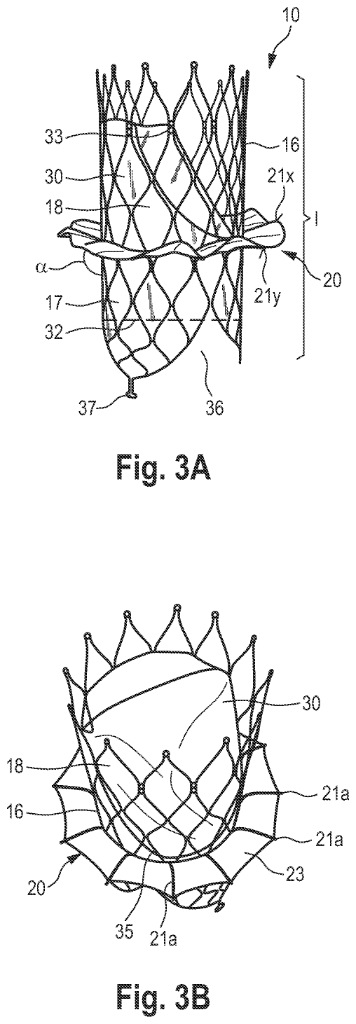

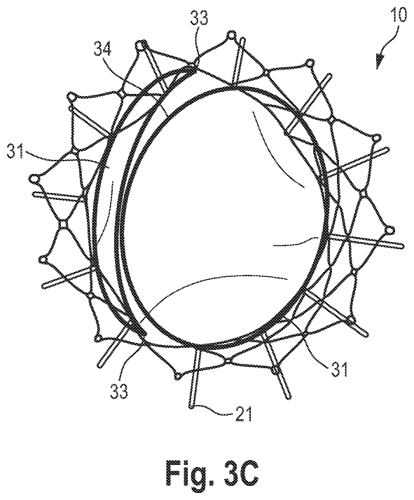

As can be seen in FIG. 2, an exemplary device 10 according to the invention is placed in the expanded state in the human heart 50. The device as such is shown in more detail in FIG. 3, and in the following it will be made reference to both, FIGS. 2 and 3; for the sake of better understanding, not all of the features of the device designated in FIG. 3 are designated in FIG. 2, however, the features are nevertheless the same.

The prosthetic device 10 comprises an expandable stent frame 16 having a length l, an interior area 11, and an exterior area 12. The stent frame 16 comprises a proximal portion 17 configured for placement in the vena cava inferior 53 (see FIG. 2A) and a distal portion 18 configured for protruding from the vena cava inferior 53 into the right atrium 54 (see FIG. 2A).

The prosthetic device further comprises a valve element 30 attached to the stent frame 16 in the distal portion 18.

As can be seen in FIG. 2A and FIG. 3 there is provided an annular positioning assembly 20 coupled to the stent frame 16 and extending outwardly and radially from the exterior area 12 of the stent frame 16. The annular positioning assembly comprising a plurality of outwardly radially expanding positioning members 21 and a cover member 23 covering the positioning members 21. The annular positioning assembly 20, at the stent frame 16, is provided between the distal portion 18 and the proximal portion 17, and is configured for positioning the distal portion 18 of the stent frame 16 in the right atrium 54 and the proximal portion 17 in the vena cava inferior 53.

In the example shown in FIG. 3, the outwardly radially expanding positioning members 21 of the positioning assembly 20 represent stent wire members in the form of struts 21a. Alternatively, the positioning members 21 can represent stent wire members that are V-shaped wire portions. The number of the stent wire members/positioning members 21 representing struts 21a or V-shaped wire portions may depend on the device 10 to be implanted, as well as on the special circumstances of the dimensions and specifications of the heart portion of a patient to be treated, and preferably between 3 and 15 positioning members 21 are present.

As can also be seen, e.g. from FIG. 3, the positioning members 21 extend outwardly from the stent frame 16 at an angle α, which is, in the example shown in FIG. 3, about 90 degrees, and can be between about 60 and 150 degrees (including about 60 and about 150), preferably between about 80 and about 120 degrees, including about 80 and about 120 degrees, preferably about 90 degrees.

In the example shown in FIG. 3, the positioning members 21 extend outwardly from the stent frame 16 at slightly different angles, which design, herein, is designated as non-symmetrically. In other words, some struts 21a of the positioning members 21 extend outwardly at an angle of about 90 degrees, whereby some other struts 21a of the positioning members of the same positioning assembly 20 extend at about 80 degrees, and other struts 21a may extend at about 100 degrees.

As can also be seen from FIG. 3, the cover member 23 covers the positioning members 21, such, that a circumferential rim-type element, i.e. the positioning assembly is generated, which circumferentially protrudes from the longitudinal axis of the device 10 at an angle of about 90 degrees.

In the embodiment shown in FIG. 3, the stent frame 16 represents a stent frame formed from interconnected stent wires, which interconnection forms stent frame cells 35, which are about diamond-shaped, thus, forming a pattern of circumferential rows of diamond-shaped cells being adjacent to one another. At the very end of the distal portion 18, the edges of the terminal diamond-shaped cells are freely pointing in the distal direction, which means, that the terminal edges are not connected to an adjacent cell. At the very end of the proximal portion 17, in the embodiment shown in FIG. 3, the three last rows of the diamond-shaped cells are not fully circumferentially surrounding the circumference of the stent frame, but leave cell-free spaces, by means of which the borders of the diamond-shaped cells 35, at three positions around the circumference of the stent frame, form about V-shaped cut-outs 36. At three positions at the very end of the proximal portions, T-bar-like connecting struts 37 are provided.

As can be also taken from FIG. 3, the valve element 30 comprises two leaflets 31, a valve skirt 32 and plurality of valve commissure points 33. The valve element 30, in the embodiment shown in FIG. 3, has an asymmetrical leaflet design, one rigid leaflet and on flexible leaflet.

As can be also taken from FIG. 3, the valve element 30 is attached within the interior area 11 of the stent frame 16 in the distal portion 18.

It is to be noted that the stent frame, according to an embodiment of the invention, can be—at least partially—covered on its exterior area 12 by a prosthetic material, preferably only in the proximal portion 17.

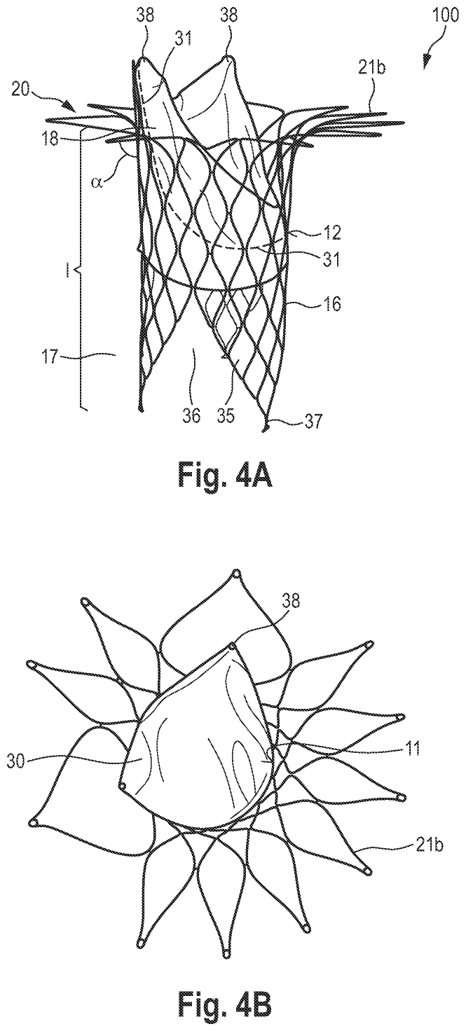

FIG. 4 shows another embodiment of the device of the invention, with the prosthetic device being designated with the reference number 100. In this embodiment and in FIG. 4, the features of the prosthetic device 100, which are otherwise the same as in the prosthetic device 10, are designated with the same reference numbers as the prosthetic device 10.

As the prosthetic device in FIG. 3, the prosthetic device 100 shown in FIG. 4 comprises an expandable stent frame 16 having a length l, an interior area 11, and an exterior area 12. The stent frame 16 comprises a proximal portion 17 configured for placement in the vena cava inferior 53 (see FIG. 2A) and a distal portion 18 configured for protruding from the vena cava inferior 53 into the right atrium 54 (see FIG. 2A), wherein the distal portion 18 is shorter in length than the distal portion 18 of the embodiment shown in FIG. 3.

The stent frame 16 comprises, at the very end of the distal portion 18, a stent crown configuration, which is bent outwardly at a certain angle, thus generating the plurality of outwardly radially expanding positioning members 21 of the annular positioning assembly, extending outwardly and radially from the exterior area of the stent frame 16.

In the embodiment shown in FIG. 4, the positioning members 21, i.e. the outwardly bound stent configuration, are not covered by a cover member; however, according to another embodiment of the invention, the positioning members 21, i.e. the V-shaped wire portions 21b, can be covered.

The prosthetic device 100 further comprises a valve element 30 attached to the stent frame 16, the valve element 30 comprising a rigid leaflet and a flexible leaflet 31, and is attached within the interior area 11 of the stent frame. The valve element 30 is attached to the stent frame 16, such, that a portion of the leaflets 31 extend beyond the end of the distal portion 18. This is achieved by attaching a leaflet 31 of the valve element 30 to one or more stent wire extensions 38 extending beyond the outwardly bound stent crown, i.e. V-shaped positioning members 21b. As can be taken from FIG. 4, the valve element 30 further comprises a valve skirt 32 and plurality of valve commissure points 33, by means of which the valve element 30 is attached to the stent wire extensions. The valve element 30, in the embodiment shown in FIG. 3, has an asymmetrical leaflet design.

In the example shown in FIG. 4, the V-shaped outwardly radially expanding positioning members, i.e. the V-shaped stent wire portions 21b, of the positioning assembly 20 represent the last row of the stent frame 16, i.e. the “stent (frame) crown”. The number of the positioning members 21 representing V-shaped wire portions may depend on the device 100 to be implanted, as well as on the special circumstances of the dimensions and specifications of the heart portion of a patient to be treated, and preferably between 3 and 15 positioning members 21 are present. In the embodiment shown in FIG. 4, 11 positioning members 21 are provided.

As can also be seen, in FIG. 4, the positioning members 21 extend outwardly from the stent frame 16 at an angle α, which is, in the example shown in FIG. 4A, about 90 degrees, wherein the angle can generally be between about 60 and 150 degrees (including about 60 and about 150), preferably between about 80 and about 120 degrees, including about 80 and about 120 degrees, preferably about 90 degrees.

Also, with this embodiment, the positioning members 21 can extend outwardly from the stent frame 16 at slightly different angles, which design, herein, is designated as non-symmetrically. In other words, some V-shaped wire portions 21b of the positioning members 21 extend outwardly at an angle of about 90 degrees, whereby some other V-shaped wire portions extend at about 80 degrees, and other struts 21a may extend at about 100 degrees.

Also in the embodiment shown in FIG. 4, the stent frame 16 represents a stent frame formed from interconnected stent wires, which interconnection forms stent frame cells 35, which are about diamond-shaped, thus, forming a pattern of circumferential rows of diamond-shaped cells 35 being adjacent to one another. At the very end of the distal portion 18, the stent crown is provided, which, as described above, is bent outwardly.

Towards the end of/in direction of the proximal portion 17, in the embodiment shown in FIG. 4, the four last rows of the diamond-shaped cells are not fully circumferentially surrounding the circumference of the stent frame, but leave cell-free spaces, by means of which the borders of the diamond-shaped cells 35, at three positions around the circumference of the stent frame, form about V-shaped cut-outs 36. At three positions at the very end of the proximal portions, T-bar-like connecting struts 37 are provided.

It is to be noted that the stent frame 16 of this embodiment, according to an embodiment of the invention, can be—at least partially—covered on its exterior area 12 by a prosthetic material, preferably only in the proximal portion 17.

What is claimed is:

1. A prosthetic device for treating venous backflow, the prosthetic device being configured for placement into the junction between right atrium and vena cava inferior of a patient and comprising
an expandable stent frame having a length l, an interior area, an exterior area, the stent frame comprising a proximal portion configured for placement in the vena cava inferior and a distal portion configured for protruding from the vena cava inferior into the right atrium,
a valve element attached to the stent frame,
an annular positioning assembly coupled to the stent frame and extending outwardly and radially from the exterior area of the stent frame, the annular positioning assembly comprising a plurality of outwardly radially expanding positioning members, wherein the annular positioning assembly, at the stent frame, is provided between the distal portion and the proximal portion, and is configured for positioning the distal portion of the stent frame in the right atrium and the proximal portion in the vena cava inferior.

2. The prosthetic device of claim 1, wherein the annular positioning assembly further comprising a cover member covering the positioning members.

3. The prosthetic device of claim 1, wherein the valve element is attached to the stent frame in the distal portion.

4. The prosthetic device of claim 1, wherein the valve element comprises one or two leaflet(s), a valve skirt, and a plurality of valve commissure points.

5. The prosthetic device of claim 1, wherein the valve element has an asymmetrical leaflet design.

6. The prosthetic device of claim 1, wherein the valve element comprises one valve leaflet and a flexible coaptation section, a valve skirt, and two valve commissure points.

7. The prosthetic device of claim 1, wherein the valve element consists of one rigid leaflet and one flexible leaflet.

8. The prosthetic device of claim 1, wherein the valve element is attached within the interior area of the stent frame in the distal portion.

9. The prosthetic device of claim 1, wherein the outwardly radially expanding positioning members of the positioning assembly are stent wire members selected from struts or V-shaped wire portions.

10. The prosthetic device of claim 1, wherein the annular positioning assembly comprises between 1 and 30, preferably between 3 and 15, outwardly radially expanding positioning members.

11. The prosthetic device of claim 1, wherein the positioning members extend outwardly from the stent frame at an angle α between 60 and 150 degrees, preferably between 90 and 120 degrees.

12. The prosthetic device of claim 1, wherein the positioning members radially extend non-symmetrically.

13. The prosthetic device of claim 1, wherein the stent frame is at least partially covered on its exterior area by a prosthetic material.

14. The prosthetic device of claim 1, wherein the stent frame is at least partially covered on its exterior area by a prosthetic material in the proximal portion only.

15. The prosthetic device of claim 1, wherein the stent frame is at least partially covered on its exterior area by a prosthetic material, such, that the height of a covered section varies around the circumference of the stent frame.

16. The prosthetic device of claim 1, wherein the positioning members have two faces, one face facing towards the distal portion, one face facing towards the proximal portion, and in that the cover member covering the positioning members is attached to the positioning members on either or both of the two faces of the positioning members.

17. The prosthetic device of claim 1, wherein the cover member covering the positioning members comprises or consists of a material selected from a biological source material or a synthetic material.

18. The prosthetic device of claim 1, wherein the cover member comprises or consists of a material that is selected from pericardial tissue, polyester, polyurethane, polystyrene, polytetrafluorethylene, ultra-high-molecular-weight polyethylene (UHMPE), and mixtures thereof.

19. The prosthetic device of claim 1, wherein the device comprises radiopaque markers, preferably placed in an asymmetric order onto the stent frame or onto the cover member.

20. A method for treating tricuspid regurgitation, the method comprising the step of placing the prosthetic device of claim 1 into the vena cava inferior of a patient in need of treatment of the tricuspid regurgitation, such, that the proximal portion of the prosthetic device is placed in the vena cava inferior, and that the distal portion of the prosthetic device protrudes from the vena cava inferior into the right atrium, wherein the annular positioning assembly positions the distal portion of the main body in the right atrium and the proximal portion in the vena cava inferior.
